(19) United States
(12) Patent Application Publication
(10) Pub. No.: US 2024/0266062 A1
(43) Pub. Date: Aug. 8, 2024

(54) DISEASE RISK EVALUATION METHOD, DISEASE RISK EVALUATION SYSTEM, AND HEALTH INFORMATION PROCESSING DEVICE

(71) Applicants: RIKEN, Wako-shi, Saitama (JP); SAI Corporation, Wako-shi, Saitama (JP); Topcon Corporation, Itabashi-ku, Tokyo (JP)

(72) Inventors: Satoshi WADA, Wako (JP); Kei TANEISHI, Wako (JP); Yasufumi FUKUMA, Wako (JP); Zaixing MAO, Wako (JP); Hisashi TSUKADA, Wako (JP)

(21) Appl. No.: 18/562,777

(22) PCT Filed: May 27, 2022
(86) PCT No.: PCT/JP2022/021744

§ 371 Date: Nov. 20, 2023

(30) Foreign Application Priority Data
May 28, 2021 (JP) 2021-090157
Sep. 17, 2021 (JP) 2021-152523

(51) Int. Cl. G16H 50/30

(52) U.S. Cl. CPC G16H 50/30

(57) ABSTRACT

Provided are a disease risk evaluation method, a disease risk evaluation system and a health information processing device, whereby it becomes possible to detect the latent onset tendency in a healthy stage in advance and to quantify the prospective disease risk of a disease of interest. Each of the disease risk evaluation method, the disease risk evaluation system and the health information processing device according to the present invention includes a plurality of steps, i.e., a step for classifying into a group in which the susceptibility to developing a specific disease is high and a group in which the susceptibility to developing the specific disease is low regardless of the degree of progression of the disease from a healthy stage until the onset of the disease, and a step for further classifying the degrees of the development of the disease in a group in which the incidence risk is determined as high. Each of the disease risk evaluation method, the disease risk evaluation system and the health information processing device is characterized by being achieved by changing the type of data to be used in a data-driven analysis in each of the steps.

### TECHNICAL FIELD

The present invention relates to a disease risk evaluation method, a disease risk evaluation system, and a health information processing device for determining whether the risk of developing a specific disease is high or not in a healthy stage.

### BACKGROUND ART

Accompanying the recent development of advanced medical care, treatment and medical techniques after the onset of a disease is advancing. As a result, life expectancy is increasing, but medical expenses for the whole nation are increasing. The financial burden for the medical expenses has become a serious social problem. In addition to physical diseases, mental health problems such as depression due to stress, and the necessity of control and improvement of lifestyles that lead to unhealthiness are also pointed out.

In order to reduce the medical expenses and enable the people to work in good health, that is, in order to extend healthy life expectancy, it is required to manage the incidence risk in a healthy stage where disease has not appeared yet and realize very early health management to prevent approach to the onset of disease. For this purpose, it is necessary to know which disease one has a high risk of developing before symptoms appear, in a healthy stage.

Indicators (criteria for measured values) used for a medical checkup such as multiphasic health screening and disease diagnosis indicate that signs of the onset of a disease have appeared and do not give criteria for risks in a healthy stage. Therefore, a new indicator that is effective in a healthy stage is required.

At present, genes and genetic mutations are indicators about which disease one is susceptible to. It is, however, known that gene expression differs depending on environmental factors. Further, in many cases, there are many gene mutations that are said to be related to a specific disease, not just one. Therefore, it is difficult to clearly know which disease incidence risk is high and which disease is approaching the onset stage or not from a current health state, only with gene information.

If it is possible to know which disease one is susceptible to without using gene information by analyzing big data, which disease the risk of developing is high is known in a healthy stage. Further, if the determination is made with measured values that change depending on a health state, it is possible to analyze at which measured value the incidence risk decreases. There is required a method for determining whether the risk of developing a specific disease is high or not irrespective of whether a healthy stage or a stage after the onset, with an environment around a specific individual and measured values, without depending on gene information.

In order to manage an incidence risk in a healthy stage where disease has not appeared yet and realize very early health management to avoid approaching the onset of the disease, it is thought to be necessary that the following requirements are satisfied.

One is to be able to determine whether the risk of developing a specific disease is high or not with measured values related to health, which are used for diagnosis of the disease, medical checkup, and the like, before signs of developing the disease appear; and one more is to be able to know degrees from before appearance of the signs of incidence until the onset of the disease.

### CITATION LIST

### Patent Literature

PATENT LITERATURE 1: JP-A-2013-191020

### SUMMARY OF INVENTION

### Technical Problem

In Patent Literature 1, though it is said that a state is estimated using a self-organizing map technique, only description on very common unsupervised learning is made, and effects of applying the unsupervised learning is not described. Further, since only classification of medical checkup data is performed, it is not possible to find out an incidence risk in a healthy stage, which we attempt to achieve.

The method that does not include inference over time cannot be said to have realized a very early health management technique in order to manage an incidence risk and avoid approaching the onset of disease. Furthermore, validation over time is also required.

We develop a technique for determining, for a person in a healthy stage, whether the risk of developing a specific disease is high or low from various environment data including medical checkup data, without using gene information. Furthermore, even in a healthy stage, whether one is approaching an incidence risk or not is quantified.

We devised a method for achieving the two requirements described before and a system using the method.

(1) To be able to determine whether the risk of developing a specific disease is high or not with measured values used for disease diagnosis, medical checkup, and the like, before signs of developing the disease appear.

It is conceivable to realize this function by the so-called data-driven analysis, and Patent Literature 1 also adopts such an approach. In a healthy stage, a supervised learning method cannot be used because biomarkers indicating diagnosis and symptoms thereof do not exist. As for the self-organizing learning self-organizing map used in Patent Literature 1, however, mapping is performed according to whether there are symptoms or not, that is, whether the risk of a specific disease has appeared or not. Therefore, the map cannot be used to determine whether the risk of developing a specific disease is high or not before symptoms appear or determine whether one is approaching the onset of the disease or not before symptoms appear.

That is, Patent Literature 1 only says that mapping was performed using a self-organizing map that is already known in the world, and does not satisfy the function as a method to realize our purpose. Further, a device for realizing our purpose is not shown.

(2) To be able to know degrees from before appearance of the signs of developing the disease until the onset of the disease.

Not only in Patent Literature 1, a method of determining the degree of the risk of developing a specific disease in a healthy stage, from medical checkup data has not been devised. It has been known in recent studies that genetic screening is much influenced by acquired change in gene expression and environmental factors, and it is important to determine the degree of an incidence risk from medical checkup data.

Therefore, an object of the present invention is to provide a disease risk evaluation method, a disease risk evaluation system, and a health information processing device capable of detecting a latent onset tendency in a healthy stage in advance and quantifying the prospective risk of a target disease.

### Solution to Problem

We devised a way to achieve the object by taking a plurality of steps, that is, a step of classifying people into a high incidence risk group and a low incidence risk group, the groups including people who are in a healthy stage where an incidence risk has not appeared yet and people who have developed disease, and a step of further performing classification of degrees of incidence in the group determined to have a high incidence risk. Further, it was also devised to realize each step by changing types of data to be used for data-driven analysis, and validation was performed.

Specifically, clustering by unsupervised learning is performed in which data the values of which change according to the degree of progression of a disease (biomarkers used to determine the disease) is removed to classify the people into the high incidence risk group and the low incidence risk group. By removing the data that changes according to the degree of progression of the disease, both people with the disease that has progressed and people in a healthy stage can be included in one group by classifying the people according to incidence risks.

Next, the data that changes according to the degree of progression of the disease is returned, and the degree of progression (whether the risk of developing the disease and appearance of symptoms is high or low) is divided into stages or quantified. This can be realized by a conventional supervised learning technique.

In a conventional progression degree determination method, existing biomarkers did not apply to progressions of all patients. By applying the existing biomarkers to the high-risk group, it becomes possible to more accurately grasp and manage progression situations.

Since data-driven analysis is adopted, details of a mechanism between individual pieces of data and results cannot be shown. However, it was validated that our method is effective by using data that is publicly available. It can be said that this validation shows that our device is an effective method for solving the problem.

### Advantage Effects of Invention

According to the present invention, it is possible to determine whether the risk of developing a specific disease is high or not in a healthy stage where symptoms have not appeared yet, as seen from a result of the validation. Further, it is possible to obtain sequential degrees of the risk of developing the disease (disease scores).

Other objects, characteristics, and advantages of the present invention will be apparent from the following description of an embodiment of the present invention about accompanying drawings.

### BRIEF DESCRIPTION OF DRAWINGS

FIG. 1 is a diagram showing evaluation steps of a disease risk evaluation method according to one embodiment of the present invention.

FIG. 2 is a diagram showing an example of displaying scores.

FIG. 3A shows graphs showing validation results.

FIG. 3B shows graphs showing validation results.

FIG. 4 is a conceptual diagram of the disease risk evaluation method according to the present embodiment.

FIG. 5 is a diagram showing a flow of filtering processing of data used when a target disease is cardiovascular disease.

FIG. 6 is a list of parameters shown in FIG. 5.

FIG. 7 is a diagram showing a flow of filtering processing of data used when the target disease is diabetes.

FIG. 8 is a list of parameters shown in FIG. 7.

FIG. 9 is a diagram showing a flow of filtering processing of data used when the target disease is depression.

FIG. 10 is a list of parameters shown in FIG. 9.

FIG. 11 shows a cardiovascular disease subtype generation process.

FIG. 12 is a list of parameters used for cardiovascular disease subtype generation.

FIG. 13 shows cardiovascular disease subcategory analysis.

FIG. 14 shows an outline of glaucoma subcategory classification.

FIG. 15 shows graphs of diabetes progression rate analysis.

FIG. 16 is a conceptual diagram showing the whole of a disease risk evaluation system according to the present embodiment.

FIG. 17 is a diagram showing components related to clustering processing of the disease risk evaluation system according to the present embodiment.

FIG. 18 is a diagram showing components related to mapping processing of the disease risk evaluation system according to the present embodiment.

FIG. 19 is a diagram showing components related to validation processing of the disease risk evaluation system according to the present embodiment.

### DESCRIPTION OF EMBODIMENT

An embodiment of a disease risk evaluation method of the present invention will be described below.

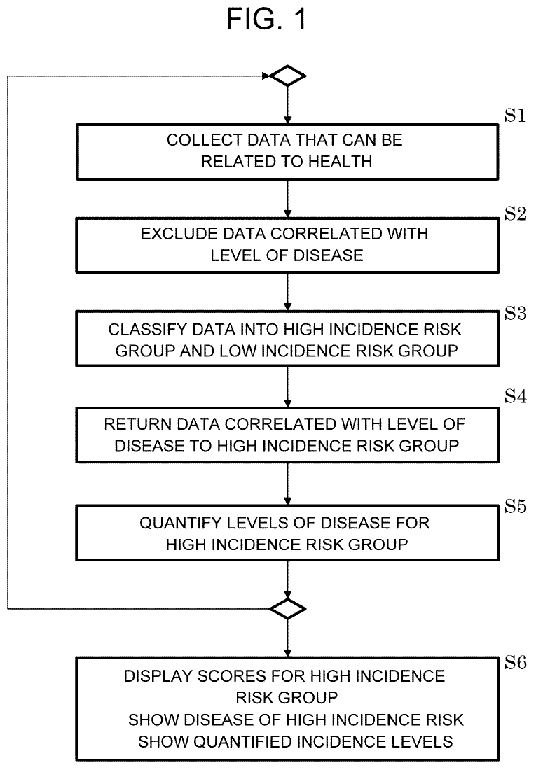

FIG. 1 shows evaluation steps according to the present embodiment.

At S1, data that can be related to health condition is collected to make a database.

It is better to collect as much data as possible as in the case of gene mutation analysis. For example, in the case of dealing with diabetes, all or a part of data of white blood cell count, lymphocyte percentage, red blood cell count, platelet count, HDL cholesterol, creatinine, albumin, height, systolic blood pressure, and medical history is used; in the case of dealing with cardiovascular disease, all or a part of data of a white blood cell count, lymphocyte percentage, red blood cell count, platelet count, HbA1c, creatinine, albumin, height, systolic blood pressure, and hours of sleep is used; and, in the case of dealing with depression, all or a part of data of a white blood cell count, lymphocyte percentage, HbA1c, total/HDL cholesterol, creatinine, albumin, height, systolic blood pressure, hours of sleep, and medical history is used. As exemplified, favorable evaluation can be obtained by using at least ten pieces of data for each disease. A part of data items may be replaced with items exemplified above. If a part of the exemplified items does not exist, other items existing as data can be used.

At S2, data correlated with disease levels (feature values) are excluded. For example, in the case of dealing with diabetes, at least HbA1c, which is used for determination of diabetes, is excluded. Thus, at S2, the data correlated with disease levels (the feature values) are excluded. By the step of S2, a database in which the data correlated with disease levels (the feature values) are not included is obtained.

At S3, data is separated into a high incidence risk group and a low incidence risk group, using the database in which the data correlated with disease levels (the feature values) are not included. For the separation at S3, a semi-supervised clustering technique is appropriate. Unsupervised clustering may be used.

By performing clustering such that cases of the onset of the disease are included, the high incidence risk group can be extracted.

At S4, the data correlated with disease levels (the feature values) are returned to the high incidence risk group. That is, the data excluded at the process of separating the data into the high incidence risk group and the low incidence risk group (S3), for example, HbA1c excluded in the case of diabetes is returned.

Then, at S5, for the high incidence risk group, disease levels from a healthy stage until after the onset of the disease are quantified, including the data correlated with the levels of the disease (the feature values).

At S5, supervised learning is appropriate. By supervised learning, it is possible to perform quantification where data does not actually exist.

When the process up to S5 has been performed for the specific disease, the process from S1 to S5 is performed for the next target disease. Thus, for all the targeted diseases, classification into groups with high and low incidence risks and quantification of disease levels are performed.

When the process ends for all the targeted diseases, scores of individual subjects are created at S6. Scores are displayed for the high incidence risk group, and quantified incidence levels showing diseases the incidence risk of which is high are displayed. The scores are numerically displayed, for example, with values from 0 to 100 inclusive. As a graphical method for displaying the scores, a bar chart or a radar chart can be used.

By S6, a person who takes this examination can know the name of a disease that he has to be careful of and the degree of the risk of developing the disease.

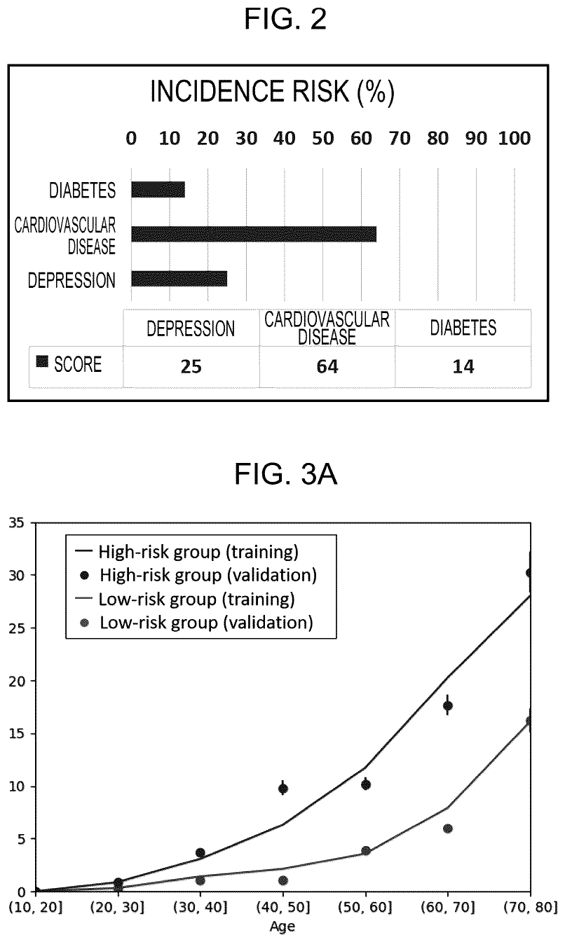

FIG. 2 shows an example of displaying the scores.

The example is an example of display to be outputted when the present evaluation method and the present evaluation system are implemented in a health information processing device. In the example, “diseases the incidence risk of which is determined to be high” and “levels from before appearance of symptoms until the onset of the diseases”, which are requirements for realizing the very early health management described before, are shown.

### Validation Method

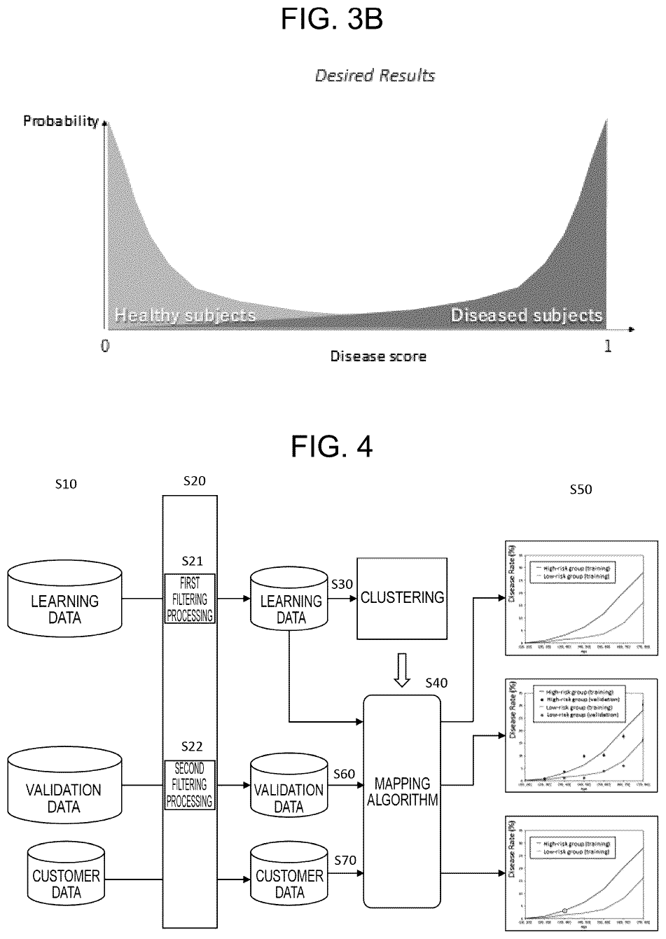

FIGS. 3A and 3B show validation results. FIG. 3A shows a validation result about the classification into the groups by S3, and FIG. 3B shows a validation result showing disease degrees for the group classified as having a high risk.

Training was performed with published data (CDC (Centers for Disease Control and Prevention) NHANES 2013-2014), and validation was performed with different data (CDC NHANES 2011-2012) that is similarly published. Thus, validation was performed by preparing a data set different from the data used for the training. In FIG. 3A, validations are indicated by dots (●).

A red solid line in FIG. 3A indicates a mean value of incidence rates of people classified as having a high risk, as a result of performing clustering with data that has been trained for cardiovascular disease, by age.

It is seen that, from aged people with a high incidence rate to young people with a low incidence rate, the people are continuously separated in a high-risk group and a low-risk group. From this, it is known that even young people who are still healthy can be separated into the high-risk and low-risk groups. This means that it is possible to predict that the possibility of developing cardiovascular disease in the future is strong by making an inference from current environmental values (measured values).

Knowledge obtained by training overlaps with the solid line, and shows that it is also effective for other data.

FIG. 3B shows disease degrees from a healthy stage until after the onset of the disease (disease scores) obtained by returning data excluded at the separation step to the group classified as having a high risk after the separation and performing supervised learning. Data-driven analysis in which biomarkers indicating symptoms that are used for diagnosis are main explanatory variables has been performed until now. Therefore, it has not been possible to analyze degrees before appearance of symptoms. In the present invention, however, semi-supervised clustering is used in a stage before appearance of symptoms, and supervised learning and data the values of which change according to degrees of disease are used in a stage of appearance of symptoms. Therefore, it is possible to sequentially show disease degrees (disease scores) from a healthy stage until after the onset of the disease.

FIG. 4 is a further detailed conceptual diagram before classification into groups (S1 to S3) in the disease risk evaluation method of the present embodiment.

In the disease risk evaluation method according to the present embodiment, at least two kinds of category data among blood test data, physical measurement data, demographic data, medical interview data, and urinalysis data are used to perform clustering into at least two groups, and a disease risk is estimated for an estimation target person who is in a healthy stage by determining which group the estimation target person belongs to or is close to. Data from which disease parameters used for diagnosis of a target disease or used for determination of progression of the target disease are excluded is used.

As shown in FIG. 4, in the disease risk evaluation method according to the present embodiment, a computer has a learning data acquisition step S10 of acquiring at least two kinds of category data, a filtering processing step S20 of removing particular parameters from the data, a learning step S30 of performing machine learning using the data from which the particular parameters have been removed, a mapping processing step S40 for displaying a result of clustering, and a display step S50 of displaying groups clustered by the learning step S30 and a determination result.

The filtering processing step S20 has a first filtering processing step S21 and a second filtering processing step S22.

At the first filtering processing step S21, for a target disease set in advance, disease parameters used for diagnosis of the target disease or determination of progression of the target disease are excluded from the data.

At the filtering processing step S22, display parameters used for displaying a result of clustering, one of parameters that are strongly correlated with each other, and parameters that decrease clustering performance are excluded.

At the learning step S30, such parameters that clustering by disease risk is, for example, separation into a low-risk group and a high-risk group are heuristically learned.

At the mapping processing step S40, for example, mapping with two axes of disease risk rate and age distribution is performed.

At the display step S50, the low-risk group and the high-risk group are two-dimensionally displayed with line graphs, for example, with age distribution and disease risk indicated by the X and Y axes, respectively.

The computer has a validation step S60 of performing validation of the groups clustered by the learning step S30.

At the learning step S30, data during a first predetermined period in the past is used as learning data. At the validation step S60, data during a second predetermined period before the first predetermined period is used as validation data. For example, CDC (Centers for Disease Control and Prevention) 2013-2014 data is used as the learning data, and CDC 2011-2012 data is used as the validation data.

As for the validation data used at the validation step S60, disease parameters are excluded by the first filtering processing step S21, and display parameters used to display a result of clustering or one of parameters that are strongly correlated with each other, and parameters that decrease the clustering performance are excluded by the second filtering processing step S22.

At the display step S50, by displaying the low-risk and high-risk groups with plots, consistency with the line graphs is displayed.

The computer has a determination step S70 of determining, for the estimation target person, which group the estimation target person belongs to or is close to.

For target person data of the estimation target person used at the determination step S70, disease parameters are excluded by the first filtering processing step S21, and display parameters used to display a result of clustering, one of parameters that are strongly correlated with each other, and parameters that decrease the clustering performance are excluded by the second filtering processing step S22.

At the display step S50, by displaying a determination result about the estimation target person with a plot, the determination result can be compared with the line graphs of the low-risk and high-risk groups, and it is possible to determine which group the estimation target person is close to, that is, a risk position. Further, it is possible to evaluate a risk after many years from the distribution for each age group.

As for parameters used at the learning step S30, the validation step S60, and the determination step S70, especially, gender, age group, and medical interview, it is preferable to normalize and use the parameters. For example, the parameters are normalized with SD values and used.

In the clustering by disease risk, it is preferable to extract the group with a high risk of a target disease as one group, including a healthy stage, the onset of the disease, and a progression stage, and perform grading according to degrees of progression for the extracted group.

Further, in the clustering by disease risk, a Kernel k-means method or an independent kernel function can be used. For example, initialization (center point setting) is performed for 40% of learning data with disease labels, and clustering about whether a high risk and a low risk for each age group at the center point (each non-disease category) is performed.

At the validation step S60, validation can be performed with teaching data used at the learning step S30. The validation can be performed by inputting validation data to a constructed clustering model and comparing a result of the learning data with an error of the prevalence rate of the disease risk. Further, validation can be performed from the past histories of those who have developed the disease.

Thus, by performing machine learning using data from which disease parameters used for diagnosis of the target disease or used for determination of progression of the target disease are excluded, it is possible to detect a latent tendency of the onset of the target disease in a healthy stage in advance and quantify the prospective risk of the target disease.

Then, by analyzing lifestyles of the high disease risk group and the low disease risk group, it is possible to realize an application enabling health promotion management and show intervention guidelines for reducing the disease risk.

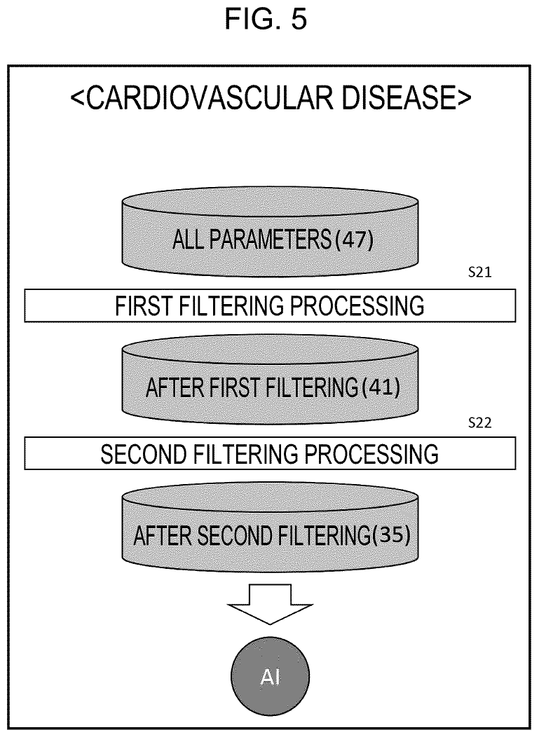

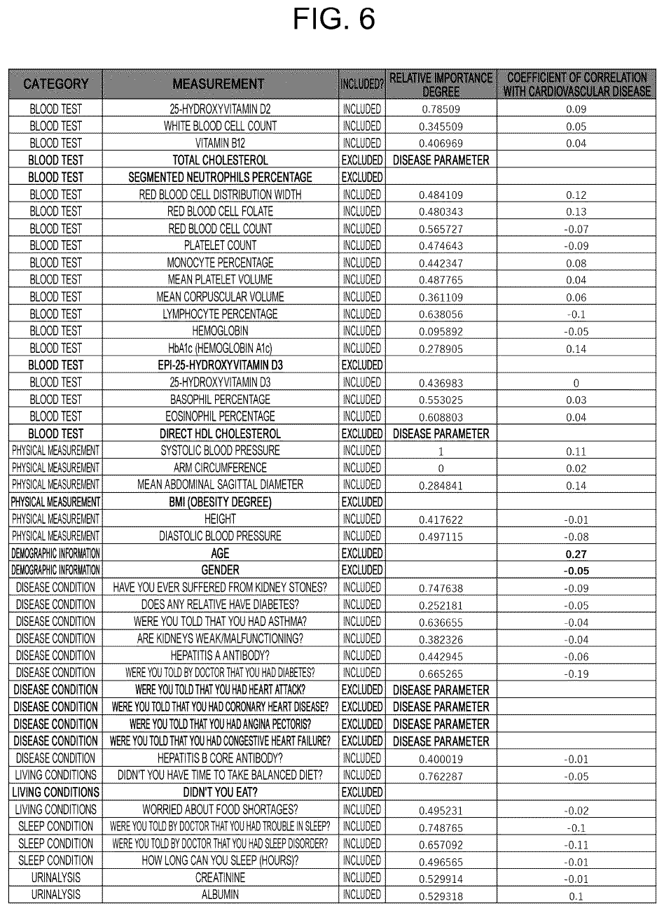

FIG. 5 shows a flow of filtering processing of data used when the target disease is cardiovascular disease, and FIG. 6 is a list of parameters shown in FIG. 5.

As shown in FIG. 5, when the target disease is cardiovascular disease, six parameters are excluded at the first filtering processing step S21, and six parameters are further excluded at the second filtering processing step S22.

At the first filtering processing step S21, total cholesterol and direct HDL cholesterol, which are blood test data, among the parameters shown in FIG. 6 are excluded as disease parameters. Further, at the first filtering processing step S21, medical interview parameters about the present or past diseases of the estimation target person of having heart attack, coronary heart disease, angina pectoris, or congestive heart failure, which are medical interview data, among the parameters shown in FIG. 6 are excluded as disease parameters.

At the second filtering processing step S22, segmented neutrophils percentage and epi-25-Hydroxyvitamin D3, which are blood test data, among the parameters shown in FIG. 6 are excluded, and BMI, which is physical measurement data, is excluded. This is because the segmented neutrophils percentage enhances the clustering performance, epi-25-Hydroxyvitamin D3 is strongly correlated with 25-Hydroxyvitamin D3, and BMI is strongly correlated with mean abdominal sagittal diameter.

Further, at the second filtering processing step S22, among the parameters shown in FIG. 6, age and gender parameters, which are demographic data, are excluded, and a medical interview parameter of “Didn't you eat?”, which is medical interview data, is excluded.

This is because gender enhances the clustering performance, and the medical interview parameter of “Didn't you eat?” is strongly correlated with a medical interview parameter of “Didn't you have time enough to take a balanced diet?”.

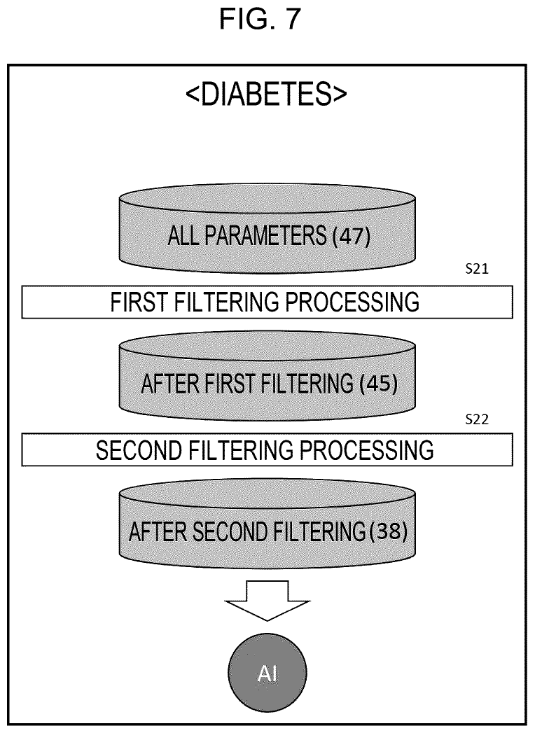

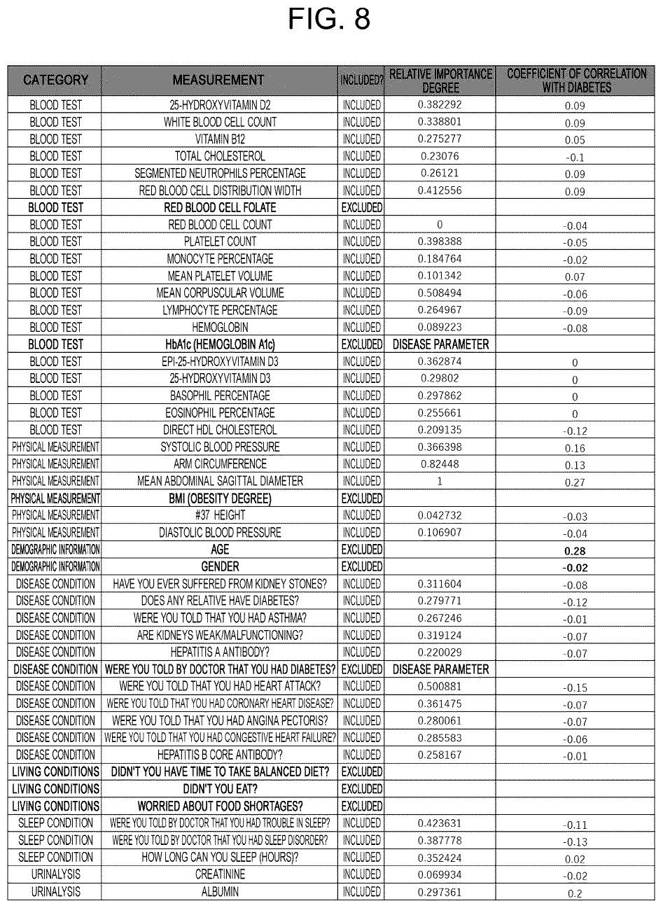

FIG. 7 shows a flow of filtering processing of data used when the target disease is diabetes, and FIG. 8 is a list of parameters shown in FIG. 7.

As shown in FIG. 7, when the target disease is diabetes, two parameters are excluded at the first filtering processing step S21, and seven parameters are further excluded at the second filtering processing step S22.

At the first filtering processing step S21, HbA1c, which is blood test data, among the parameters shown in FIG. 8 are excluded as a disease parameter. Further, at the first filtering processing step S21, a medical interview parameter about the present and past diseases of the estimation target person of having diabetes, which is medical interview data, among the parameters shown in FIG. 8 is excluded as a disease parameter.

At the second filtering processing step S22, red blood cell folate, which is blood test data, among the parameters shown in FIG. 8 is excluded, and BMI, which is physical measurement data, is excluded. This is because red blood cell folate enhances the clustering performance, and BMI is strongly correlated with mean abdominal sagittal diameter.

Further, at the second filtering processing step S22, among the parameters shown in FIG. 8, age and gender parameters, which are demographic data, are excluded, and medical interview parameters of “Didn't you have time enough to take a balanced diet?”, “Didn't you eat?”, and “Are you worried about food shortages?”, which are medical interview data, are excluded.

This is because gender and the medical interview parameters enhance the clustering performance.

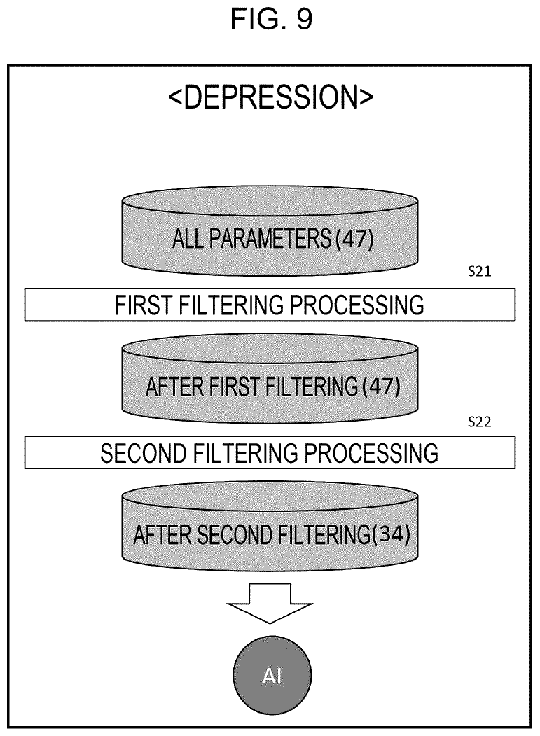

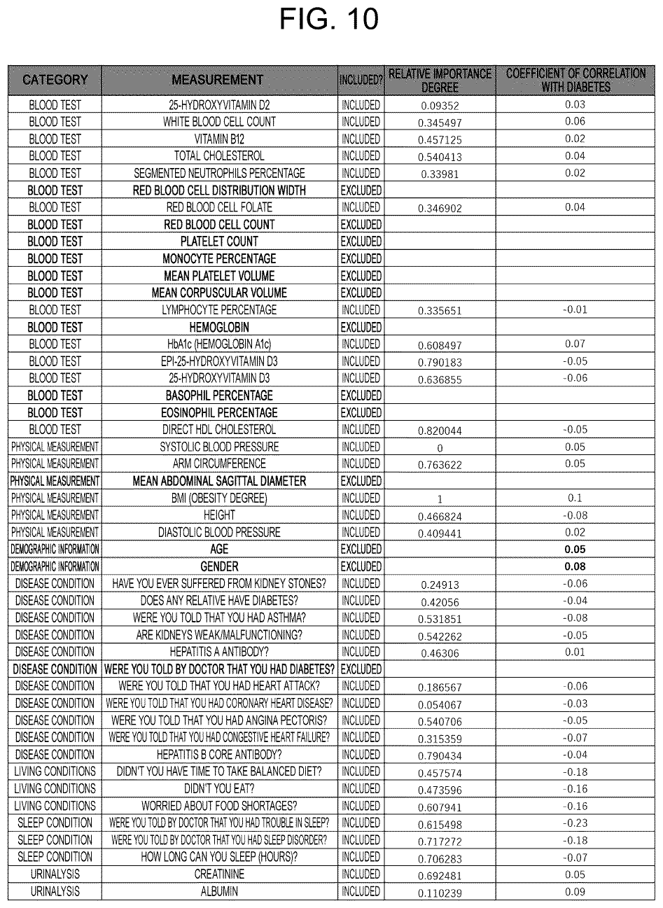

FIG. 9 shows a flow of filtering processing of data used when the target disease is depression, and FIG. 10 is a list of parameters shown in FIG. 9.

As shown in FIG. 9, when the target disease is depression, there are no parameters to be excluded at the first filtering processing step S21, and thirteen parameters are excluded at the second filtering processing step S22.

At the second filtering processing step S22, red blood cell distribution width, red blood cell count, platelet count, monocyte percentage, mean platelet volume, mean corpuscular volume, hemoglobin, basophil percentage, and eosinophil percentage, which are blood test data, among the parameters shown in FIG. 10, are excluded, and mean abdominal sagittal diameter, which is physical measurement data, is excluded. This is because red blood cell distribution width, red blood cell count, platelet count, monocyte percentage, mean platelet volume, mean corpuscular volume, hemoglobin, basophil percentage, and eosinophil percentage enhance the clustering performance, and mean abdominal sagittal diameter is strongly correlated with BMI.

At the second filtering processing step S22, age and gender parameters, which are demographic data, among the parameters shown in FIG. 10 are excluded, and a medical interview parameter of “Were you told by a doctor that you have diabetes?”, which is medical interview data, is excluded.

This is because gender and the medical interview parameter enhance the clustering performance.

In the present embodiment, when the target disease is cardiovascular disease, blood test data, physical measurement data, medical interview data, and urinalysis data are used as category data, and thirty-five parameters in the category data are used; when the target disease is diabetes, the blood test data, the physical measurement data, the medical interview data, and the urinalysis data are used as category data, and thirty-eight parameters in the category data are used; and, when the target disease is depression, the blood test data, the physical measurement data, the medical interview data, and the urinalysis data are used as category data, and thirty-four parameters in the category data are used. However, only any of the pieces of category data may be used, and it is preferable to use at least two pieces of category data. Especially, by not using the category data of the blood test data, it is possible to estimate a disease incidence risk in a health stage without conducting a highly invasive and infiltrative test accompanied by mental pain.

As for the number of parameters, any number of parameters can be used.

For example, when the target disease is cardiovascular disease, total cholesterol and direct HDL cholesterol are excluded from determination data as disease parameters if they are included as blood test data, but, if 25-hydroxyvitamin D2, white blood cell count, vitamin B12, segmented neutrophils percentage, red blood cell distribution width, red blood cell folate, red blood cell count, platelet count, monocyte percentage, mean platelet volume, mean corpuscular volume, lymphocyte percentage, hemoglobin, HbA1c, epi-25-Hydroxyvitamin D3, 25-Hydroxyvitamin D3, basophil percentage, or eosinophil percentage as a blood test parameter is included as blood test data, then at least one blood test parameter can be used as determination data.

Further, when the target disease is cardiovascular disease, and systolic blood pressure, diastolic blood pressure, arm circumference, mean abdominal sagittal diameter, BMI, or height as a physical measurement parameter is included as physical measurement data, then at least one physical measurement parameter can be used as determination data.

Further, when the target disease is cardiovascular disease, and a medical interview about the estimation target person having heart attack, coronary heart disease, angina pectoris, or congestive heart failure in the present or in the past as an medical interview parameter is included as medical interview data, then the medical interview data is excluded from the determination data as a disease parameter; but, if a medical interview about kidney stone, diabetes, asthma, kidney, hepatitis, or sleep as a medical interview parameter is included as medical interview data, then at least one medical interview parameter can be used as determination data.

Further, when the target disease is cardiovascular disease, and creatinine or albumin as a urinalysis parameter is included as physical measurement data, then at least one urinalysis parameter can be used as determination data.

When the target disease is diabetes, HbA1c as a disease parameter is excluded from determination data as blood test data, but, if 25-hydroxyvitamin D2, white blood cell count, vitamin B12, total cholesterol, segmented neutrophils percentage, red blood cell distribution width, red blood cell folate, red blood cell count, platelet count, monocyte percentage, mean platelet volume, mean corpuscular volume, lymphocyte percentage, hemoglobin, epi-25-Hydroxyvitamin D3, 25-Hydroxyvitamin D3, basophil percentage, eosinophil percentage, or direct HDL cholesterol as a blood test parameter is included as blood test data, then at least one blood test parameter can be used as determination data.

Further, when the target disease is diabetes, and systolic blood pressure, diastolic blood pressure, arm circumference, mean abdominal sagittal diameter, BMI, or height as a physical measurement parameter is included as physical measurement data, then at least one physical measurement parameter can be used as determination data.

Further, when the target disease is diabetes, and a medical interview about the estimation target person having diabetes in the present or in the past as medical interview data is excluded from the determination data as a disease parameter, but, if a medical interview about kidney stone, asthma, kidney, hepatitis, heart attack, coronary heart disease, angina pectoris, congestive heart failure, or sleep as a medical interview parameter is included as medical interview data, then at least one medical interview parameter can be used as determination data.

Further, when the target disease is diabetes, and creatinine or albumin as a urinalysis parameter is included as physical measurement data, then at least one urinalysis parameter can be used as determination data.

Thus, it is possible to, by using at least one piece of category data and using determination data including any number of parameters, determine, for the estimation target person, which group he belongs to or which group he is close to, and map and display groups and a determination result at least with two axes of risk rate and age.

When the target disease is depression, and 25-hydroxyvitamin D2, white blood cell count, vitamin B12, total cholesterol, segmented neutrophils percentage, red blood cell distribution width, red blood cell folate, red blood cell count, platelet count, monocyte percentage, mean platelet volume, mean corpuscular volume, lymphocyte percentage, hemoglobin, HbA1c, epi-25-Hydroxyvitamin D3, 25-Hydroxyvitamin D3, basophil percentage, eosinophil percentage, or direct HDL cholesterol as a blood test parameter is included as blood test data, then at least one blood test parameter can be used as determination data.

Further, when the target disease is depression, and systolic blood pressure, diastolic blood pressure, arm circumference, mean abdominal sagittal diameter, BMI, or height as a physical measurement parameter is included as physical measurement data, then at least one physical measurement parameter can be used as determination data.

Further, when the target disease is depression, and a medical interview about diabetes, kidney stone, asthma, kidney, hepatitis, heart attack, coronary heart disease, angina pectoris, congestive heart failure, or sleep as a medical interview parameter is included as medical interview data, then at least one medical interview parameter can be used as determination data.

Further, when the target disease is diabetes, and creatinine or albumin as a urinalysis parameter is included as urinalysis data, then at least one urinalysis parameter can be used as the determination data.

Thus, it is possible to, by using at least one piece of category data and using determination data including any number of parameters, determine, for the estimation target person, which group he belongs to or which group he is close to, and map and display groups and a determination result at least with two axes of risk rate and age.

Relative importance degrees shown in FIGS. 6, 8 and 10 are calculated by normalizing importance degree values of all the parameters to be between 0 to 1 inclusive.

For a parameter X, a relative importance degree (X) is calculated by the following formula:

$$Relative importance degree ( X ) = ( importance degree X - minimum importance degree among all parameters ) / ( maximum importance degree among all parameters - minimum importance degree among all parameters )$$

Here, the importance degree X is:

$$Importance degree ( X ) = separation force of all parameters - separation force without X$$

The importance degree of one parameter is calculated by measuring how much separation force is influenced by deletion of the one parameter.

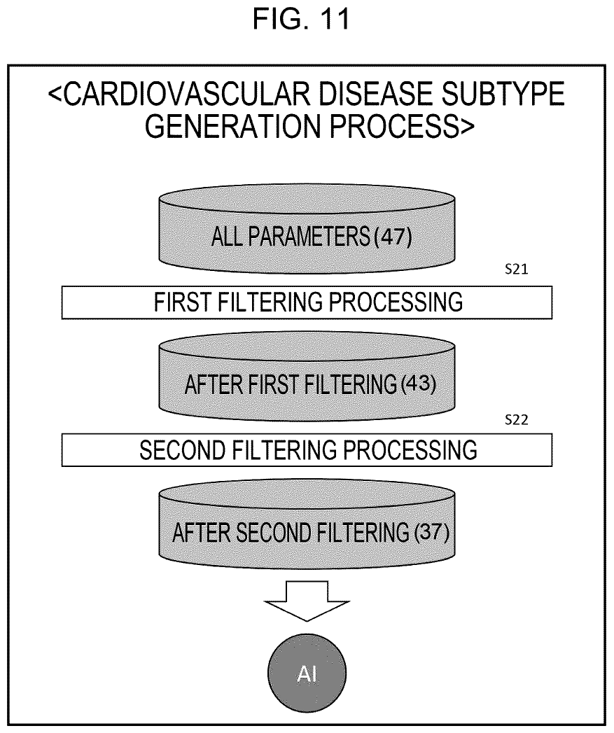

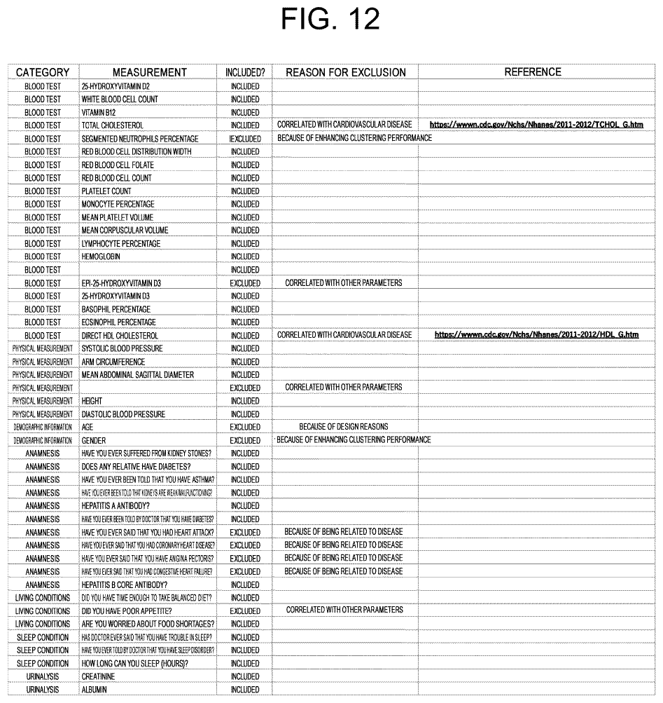

FIG. 11 is a diagram showing a cardiovascular disease subtype generation process. FIG. 11 shows a flow of filtering processing of data used when the target disease is cardiovascular disease, and FIG. 12 is a list of parameters used for the cardiovascular disease subtype generation process of FIG. 11.

As shown in FIG. 11, when the target disease is cardiovascular disease, four parameters are excluded at the first filtering processing step S21, and six parameters are further excluded at the second filtering processing step S22.

At the first filtering processing step S21, medical interview parameters of “Have you ever said that you had a heart attack?”, “Have you ever said that you have coronary heart disease?”, “Have you ever said that you had angina pectoris?”, “Have you ever said that you had congestive heart failure?”, which are medical interview data, are excluded.

At the second filtering processing step S22, segmented neutrophils rate and epi-25-Hydroxyvitamin D3, which are blood test data, among the parameters shown in FIG. 12 are excluded; BMI, which is physical measurement data, is excluded; age and gender parameters, which are demographic data, are excluded; and a medical interview parameter of “Did you have a poor appetite?”, which is medical interview data, is excluded. The segmented neutrophils rate, epi-25-Hydroxyvitamin D3, age, gender, and medical interview parameters are excluded because they enhance the clustering performance.

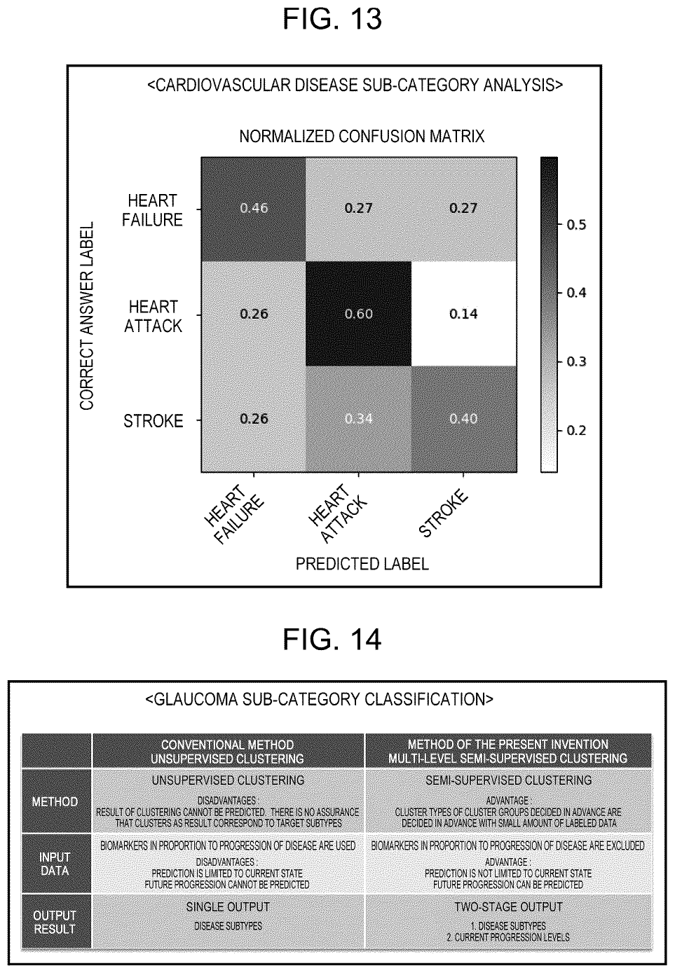

FIG. 13 is a diagram showing cardiovascular disease subcategory analysis.

A confusion matrix of FIG. 13 shows separation of various cardiovascular disease subtypes. For example, in the example of FIG. 13, if a person has had a heart attack before, the possibility of an algorithm identifying the person as a heart attack subtype is 60%, the possibility as a heart failure subtype is 26%, and the possibility as a stroke subtype is 14%.

In subcategory analysis, measured values except those of diseases indicating biomarkers are inputted as an input, and which disease subtype a patient has is outputted or displayed as an output. For a specific disease, sub-classification is further performed according to degrees of progression of the disease from a healthy stage until after the onset of the disease, and the degree of incidence in each sub-classification is displayed.

The matrix of FIG. 13 shows a validation result about classification of the cardiovascular disease subtypes. Here, consistency of data of subjects for whom diseases are actually diagnosed and categories sub-classified by AI without using the subject data is shown. In validation about the classification of the cardiovascular disease subtypes of FIG. 13, a cardiovascular risk analysis result is inputted as an input, and which cardiovascular disease subtype among heart attack, heart failure, and stroke the patient has is outputted or displayed as an output. A clustering algorithm used here is almost the same as the algorithm used for the risk analysis, but a process of the cardiovascular disease subcategory analysis is different from the process of the risk analysis in the following points. First, the outputs of the processes are different. In the risk analysis, there are only two outputs of the low risk and the high risk. In comparison, the number of outputs in the subtype classification is the same as the number of classes of subtypes. In this experiment, three subtypes of heart attack, heart failure, and stroke are considered. Second, the processes are different in teaching data (ground truth data). In the risk analysis, two kinds of labeled data for healthy subjects and for subjects with diseases are required. In comparison, in the subtype classification, labeled data is required for each disease subtype. In this experiment, three kinds of labeled data for subjects who had a heart attack, for subjects who had a heart failure, and for subjects who had a stroke are used.

FIG. 14 is a diagram showing an outline of glaucoma subcategory classification.

FIG. 14 shows difference between a glaucoma subcategory classification method according to the present invention and a conventional method using conventional unsupervised clustering. First, unsupervised clustering is performed as a clustering method in the conventional method, but semi-supervised clustering is performed in the method of the present invention. The semi-supervised clustering may be preferably multi-level semi-supervised clustering. Disadvantages of the conventional unsupervised clustering is that a result of clustering cannot be predicted and that there is no assurance that clusters as a result correspond to target subtypes. In comparison, an advantage of using the semi-supervised clustering in the method of the present invention is that cluster types of cluster groups decided in advance are decided in advance with a small amount of labeled data.

Further, though biomarkers the values of which are in proportion to progression of a disease are used as input data in the conventional method using unsupervised clustering, biomarkers the values of which are in proportion to progression of a disease are excluded in the present method. Disadvantages of using biomarkers the values of which are in proportion to progression of a disease in the conventional method are that prediction is limited to the current state of a target person and that future progression cannot be predicted. In comparison, advantages of excluding biomarkers the values of which are in proportion to progression of a disease in the method of the present invention are that prediction is not limited to the current state of a target person and that a level of progression of the current disease situation can be predicted.

Further, in the conventional method using unsupervised clustering, disease subtypes are outputted as a single output result. In comparison, in the method of the present invention, two-stage output is performed. As an output of a first stage, disease subtypes are outputted. As an output of a second stage, the current disease progression levels are outputted.

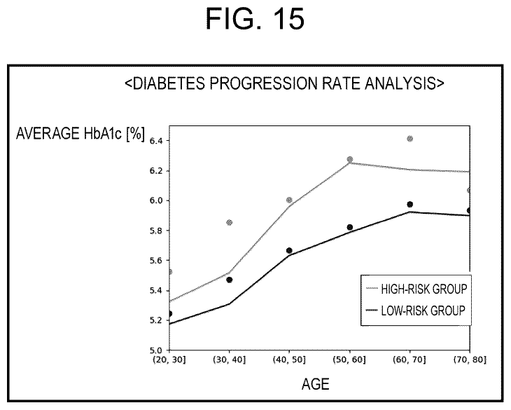

FIG. 15 is a diagram showing graphs of diabetes progression rate analysis.

In another aspect of the present invention, a step of predicting or displaying a progression speed predicted according to the degree of the risk of developing a specific disease, according to degrees of progression of the disease from a healthy stage until after the onset of the disease may be included.

The input and output of the diabetes progression rate analysis are the same as the input and output of the risk analysis, but the diabetes progression rate analysis is different from the risk analysis in the method for visualizing a result. In analysis of a risk associated with aging, the x axis represents age, and the y axis represents prevalence rate. This kind of graph shows a rate of people having a disease or the risk of the disease in various risk groups for various ages. In the progression rate analysis, the x axis represents age, and the y axis represents an average value of biomarkers indicating diseases. For example, in the case of diabetes, the y axis represents an average value of HbA1c of subjects in the same risk group with the same age. Since HbA1c is in proportion to progression of diabetes, it is shown that, the faster the change in HbA1c is, the faster the progression of diabetes is. Therefore, the slope of the progression rate analysis indicates progression rates of diabetes of subjects in various risk groups with various ages.

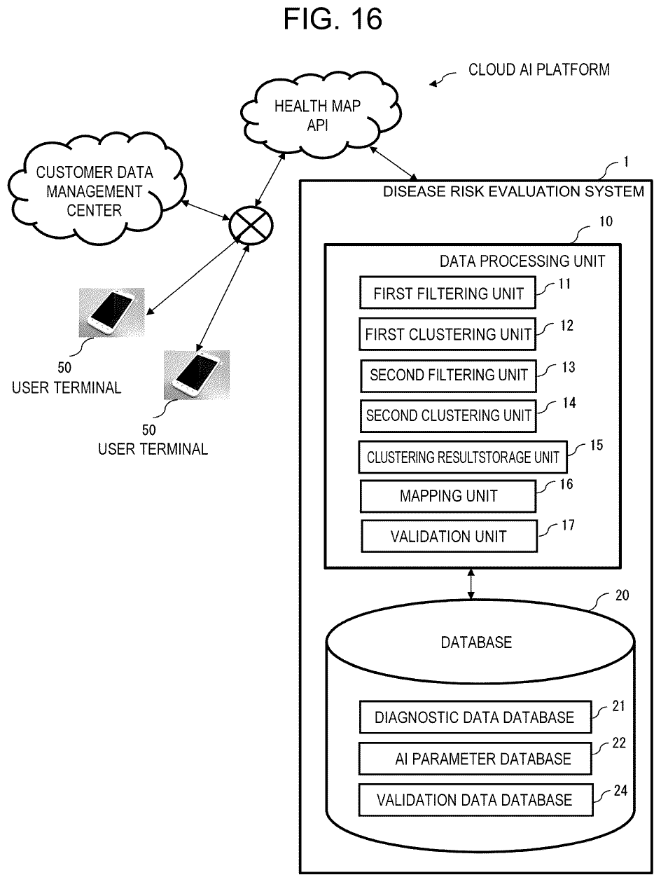

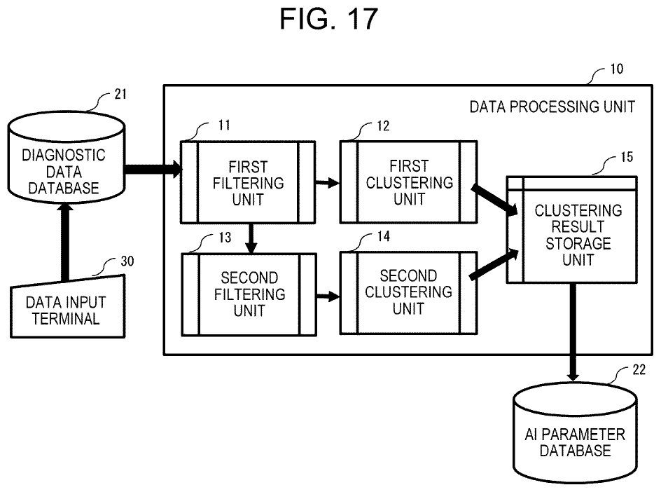

FIG. 16 is a conceptual diagram showing the whole of a disease risk evaluation system 1 according to the present embodiment.

The disease risk evaluation system 1 can be implemented as a part of a cloud AI platform. The cloud AI platform has a health map API that provides a health map to a user terminal 50 based on data inputted from a customer data management center that manages customer data of a medical institution and the like, and the user terminal 50. The disease risk evaluation system 1 of the present invention is a system for realizing the health map API and is a system that performs specific processing for generating a health map. The health map API that includes the customer data management center, the user terminal, and the disease risk evaluation system 1 is connected via a network and exchanges data.

The disease risk evaluation system 1 is provided with a data processing unit 10 and a database 20. The data processing unit 20 is provided with a first filtering unit 11, a first clustering unit 12, a second filtering unit 13, a second clustering unit 14, and a clustering model storage unit 15 for performing clustering processing. Further, the data processing unit 20 may be further provided with a mapping unit 16 for performing mapping processing. The data processing unit 20 may be further provided with a validation unit 17 that performs validation of machine learning in the clustering processing.

The database 20 includes a learning data database 21 and an AI parameter database 22 for storing data related to the clustering processing. Further, the database 20 may include a validation data database 24 for storing data related to validation of machine learning in the clustering processing.

FIG. 17 is a diagram showing components related to the clustering processing of the disease risk evaluation system 1 according to the present embodiment.

The disease evaluation system 1 for evaluating an incidence risk of a specific disease according to the present embodiment is provided with: the diagnostic data database 21 storing health-related diagnostic data; the first filtering unit 11 reading out the diagnostic data from the diagnosis database 21 and excluding diagnostic data that changes according to a level of the disease; the first clustering unit 12 performing clustering of diagnostic data that has not been excluded by the first filtering unit 11 to separate the diagnostic data into a high incidence risk group and a low incidence risk group; the second filtering unit 13 extracting only diagnostic data clustered into the high incidence risk group by the first clustering unit 12 from the diagnostic data database; the second clustering unit 14 performing clustering of the diagnostic data extracted by the second filtering unit 13 to separate the diagnostic data into a plurality of disease levels; and the clustering result storage unit 15 storing results of the clustering by the first clustering unit 12 and by the second clustering unit 14.

The diagnostic data database 21 stores health-related diagnostic data accepted from the user terminal 50 or a data input terminal 30 such as a terminal of an external system. Here, the health-related diagnostic data refers to a result of some diagnosis, medical examination, or test related to health, such as a diagnosis result obtained by health diagnosis, multiphasic health screening, or the like and a diagnosis result obtained at the time of a medical examination or test at a medical institution. The diagnostic data includes measurement items as shown in tables of FIGS. 6, 8, 10, and 12.

The first filtering unit 11 reads out the diagnostic data from the diagnosis database 21 and excludes diagnostic data that changes according to a level of a disease. That is, data correlated with levels of the disease (feature values) are excluded by the first filtering unit 11.

The first clustering unit 12 performs clustering of diagnostic data that has not been excluded by the first filtering unit 11 to separate the diagnostic data into a high incidence risk group and a low incidence risk group.

The second filtering unit 13 extracts only diagnostic data clustered into the high incidence risk group by the first clustering unit 12 from the diagnostic data database.

The second clustering unit 14 performs clustering of the diagnostic data extracted by the second filtering unit 13 to separate the diagnostic data into a plurality of disease levels.

The clustering result storage unit 15 stores a result of the clustering by the first clustering unit 12 and by the second clustering unit 14.

In the AI parameter database 22, parameters optimized by performing learning with an AI engine are stored. For example, when the AI engine is constructed with a neural network, node weights of each layer are stored in the AI parameter database 22.

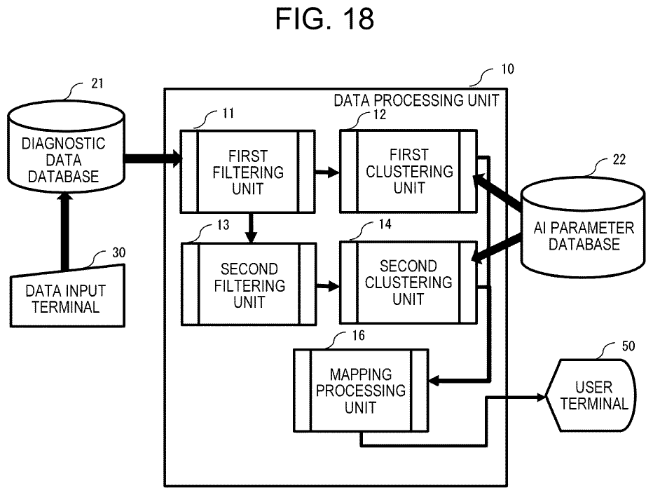

FIG. 18 is a diagram showing components related to mapping processing of the disease risk evaluation system according to the present embodiment.

As shown in FIG. 18, the disease evaluation system 1 for evaluating an incidence risk of a specific disease according to the present embodiment may be further provided with the mapping processing unit 16 that performs mapping processing for displaying a clustering result stored in the clustering result storage unit 15 as graphs.

In the diagnostic data database 21, customer data about customers, such as the customers' IDs and names, and diagnosis results about the customers' health are associated and stored. The customer data stored in the diagnostic data database 21 may be used to display a result of evaluation of an incidence risk for each customer as graphs.

The mapping processing unit 16 performs mapping processing for displaying a clustering result stored in the clustering result storage unit 15 as graphs.

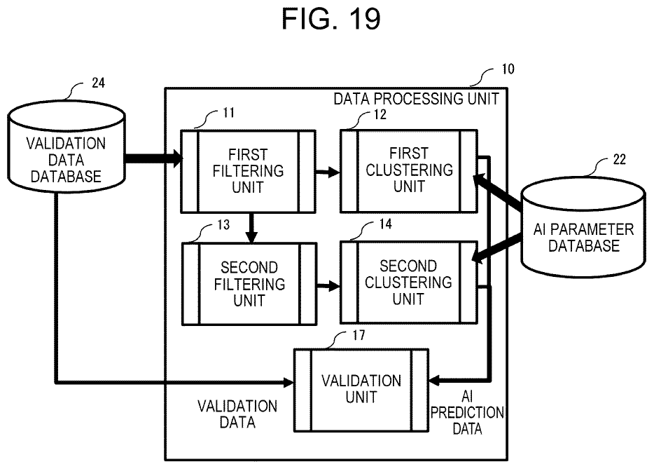

FIG. 19 is a diagram showing components related to validation processing of the disease risk evaluation system according to the present embodiment.

The disease evaluation system 1 for evaluating an incidence risk of a specific disease according to the present embodiment may be further provided with the validation unit 17 that compares validation data stored in a validation database 24 with AI prediction data which is a result of clustering by the data processing unit 10.

In the validation data database 24, the validation data is stored. The validation data, which corresponds to several years, is preferably stored in time-series.

The validation unit 17 compares the validation data stored in the validation database 24 with AI prediction data which is a result of clustering by the data processing unit 10. The AI prediction data to be compared is, for example, a result of clustering by the first clustering unit 12 or a result of clustering by the second clustering unit 14. If there is data accumulated, for example, for four years, for example, in the case of performing validation of clustering for classification into a high incidence risk group and a low incidence risk group by the first clustering unit 12, then data accumulated for the first two years is used to perform learning by AI, and validation is performed by comparing data corresponding to the second two years predicted by the AI engine with disease labels of the actual data corresponding to the second two years.

Further, for example, by comparing the degree of an incidence risk for each age group with actual incidence distribution, whether the distribution is proper or not may be validated by the verification unit 17.

### INDUSTRIAL APPLICABILITY

According to the present invention, it is possible to propose disease incidence risk reduction through intervention such as improvement of lifestyles.

According to the configuration described above, the present invention makes it possible to determine the degree of the risk of developing a specific disease from medical checkup data in a healthy stage by a method in which two stages of a first stage of classifying people, including healthy people whose incidence risk has not appeared yet (a healthy stage) and people who have already developed the disease, into a high incidence risk group and a low incidence risk group and a second stage of further performing classification of degrees of incidence in the group determined to have a high incidence risk are performed. At the first stage of classification into the high incidence risk group and the low incidence risk group, data correlated with levels of the disease (feature values), for example, HbA1c in diabetes is excluded to avoid effects of the data correlated with the levels of the disease (the feature values) on clustering. Thereby, it is made possible to perform classification into the group with a high risk of developing the specific disease and the group with a low risk, regardless of the degree of progression of the disease and before the onset of the disease. Thereby, it is possible to, for a specific disease, estimate the risk of developing the disease in a healthy stage before the onset of the disease, regardless of the state of progress of the disease, and it becomes possible to perform health management to prevent the specific disease in a healthy stage.

For example, in order to make it possible to determine the risk of developing diabetes in a healthy stage, it is realized by performing clustering, with data that changes in proportion to the degree of progression of the disease, such as HbA1c being excluded and with data of parameters that do not change with progression of the disease but accumulated as damage and can increase the risk of developing the disease in the future, such as being fat (obesity), being left.

Though the above description has been made on an embodiment, the present invention is not limited thereto, and it is apparent to one skilled in the art that various changes and modifications can be made within the scope of the principle of the present invention and accompanying claims.

### REFERENCE SIGNS LIST

S10 learning data acquisition step
S20 filtering processing step
S21 first filtering processing step
S22 second filtering processing step
S30 leaning step
S40 mapping processing step
S50 display step
S60 validation step
S70 determination step
1 disease risk evaluation system
10 data processing unit
11 first filtering unit
12 first clustering unit
13 second filtering unit
14 second clustering unit
15 mapping unit
16 comparison unit
20 database
21 learning data database
22 AI parameter database
24 validation data database
30 data input terminal
40 clustering model storage unit
50 user terminal

1. A disease risk evaluation method comprising a step of performing classification into a group of those susceptible to a specific disease and a group of those not susceptible to the specific disease, regardless of a degree of progression of the disease from a healthy stage until after onset of the disease.

2. The method according to claim 1, comprising a function of showing degrees for the group classified as having a high incidence risk.

3. The method according to claim 1, wherein both of the classification into the groups and determination of the degrees are performed, and kinds of data used for determination then are changed.

4. The method according to claim 1, wherein, in comparison with a dataset used for classification according to the degrees, a dataset used for determination of the classification into the groups according to whether the incidence risk is high or low is a dataset from which such data that values change according to the degrees are excluded to perform the classification according to the incidence risk.

5. The method according to claim 1, wherein data-driven analysis means used for determination of the classification into the groups according to whether the incidence risk is high or low is semi-supervised clustering or unsupervised clustering.

6. The method claim 1, wherein data-driven analysis means used for determination of the degrees is realized by using supervised learning and such data that values change according to the degrees.

7. The method according to claim 1, wherein gene information is not included in data sets.

8. The method according to claim 1, wherein, in presentation of whether the incidence risk is high or low and degrees of incidence, each of the degrees of incidence is normalized, and a radar chart is used to display each of the degrees.

9. A disease risk evaluation system, wherein a disease risk evaluation method comprises a step of performing classification into a group of those susceptible to a specific disease and a group of those not susceptible to the specific disease, regardless of a degree of progression of the disease from a healthy stage until after onset of the disease.

10. A disease risk evaluation system, wherein a disease risk evaluation method comprises a step of, for a specific disease, further performing sub-classification according to degrees of progression of the disease from a healthy stage until after onset of the disease, and displaying a degree of incidence for each sub-classification.

11. A disease risk evaluation system, wherein a disease risk evaluation method comprises a step of predicting or displaying a progression speed predicted according to a degree of a risk of developing a specific disease, according to degrees of progression of the disease from a healthy stage until after onset of the disease.

12. The disease risk evaluation system according to claim 9, comprising a function of showing degrees for the group classified as having a high incidence risk.

13. The disease risk evaluation system according to claim 9, wherein both of the classification into the groups and determination of the degrees are performed, and kinds of data used for determination then are changed.

14. The disease risk evaluation system according to claim 9, wherein, in comparison with a dataset used for classification according to the degrees, a dataset used for determination of the classification into the groups according to whether the incidence risk is high or low is a dataset from which such data that values change according to the degrees are excluded to perform the classification according to the incidence risk.

15. The disease risk evaluation system according to claim 9, wherein data-driven analysis means used for determination of the classification into the groups according to whether the incidence risk is high or low is semi-supervised clustering or unsupervised clustering.

16. The disease risk evaluation system according to claim 9, wherein data-driven analysis means used for determination of the degrees is realized by using supervised learning and such data that values change according to the degrees.

17. The disease risk evaluation system according to claim 9, wherein, in presentation of whether the incidence risk is high or low and degrees of incidence, each of the degrees of incidence is normalized, and a score is used to display each of the degrees.

18. A health information processing device, wherein
a disease risk evaluation method comprises a step of performing classification into a group of those susceptible to a specific disease and a group of those not susceptible to the specific disease, regardless of a degree of progression of the disease from a healthy stage until after onset of the disease; and
the health information processing device comprises a processor, the processor executing inference based on knowledge stored in a knowledge storage unit to generate disease risk evaluation information.

19. The health information processing device according to claim 18, wherein
the knowledge storage unit comprises a function of showing degrees for the group classified as having a high incidence risk.

20. The health information processing device according to claim 18, wherein the knowledge storage unit performs both of the classification into the groups and determination of the degrees, and changes kinds of data used for determination then.

21. The health information processing device according to claim 18, wherein, in comparison with a dataset used for classification according to the degrees, a dataset used by the knowledge storage unit for determination of the classification into the groups according to whether the incidence risk is high or low is a dataset from which such data that values change according to the degrees are excluded to perform the classification according to the incidence risk.

22. The health information processing device according to claim 18, wherein data-driven analysis means used by the knowledge storage unit for determination of the classification into the groups according to whether the incidence risk is high or low is semi-supervised clustering or unsupervised clustering.

23. The health information processing device according to claim 18, wherein data-driven analysis means used by the knowledge storage unit for the determination of the degrees is realized by using supervised learning and such data that values change according to the degrees.

24. The health information processing device according to claim 18, wherein, when presenting whether the incidence risk is high or low and degrees of incidence, the knowledge storage unit normalizes each of the degrees of incidence and uses a score to display each of the degrees.

25. A disease risk evaluation system (1) for evaluating an incidence risk of a specific disease, the disease risk evaluation system (1) comprising:
a diagnostic data database (21) storing health-related diagnostic data;
a first filtering unit (11) reading out the diagnostic data from the diagnosis data database (21) and excluding the diagnostic data that changes according to a level of the disease;
a first clustering unit (12) performing clustering of diagnostic data that has not been excluded by the first filtering unit (11) to separate the diagnostic data into a high incidence risk group and a low incidence risk group;
a second filtering unit (13) extracting only the diagnostic data clustered into the high incidence risk group by the first clustering unit (12) from the diagnostic data database;
a second clustering unit (14) performing clustering of the diagnostic data extracted by the second filtering unit (13) to separate the diagnostic data into a plurality of disease levels; and
a clustering result storage unit (15) storing results of clustering performed by the first clustering unit (12) and the second clustering unit (14).

26. The disease risk evaluation system according to claim 25, further comprising a mapping processing unit performing mapping processing for displaying the results of the clustering stored in the clustering result storage unit (15) as graphs.

27. The disease evaluation system according to claim 25, further comprising a validation unit (17) comparing validation data stored in a validation data database (24) and AI prediction data which is results of clustering.
